(19) United States
(12) Patent Application Publication
(10) Pub. No.: US 2007/0185399 A1
(43) Pub. Date: Aug. 9, 2007

(54) Apparatus, method, and medium for measuring body fat using a near infrared signal

(75) Inventors: Kun Kook Park, Yongin-si (KR); In Duk Hwang, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/702,514

(22) Filed: Feb. 6, 2007

(30) Foreign Application Priority Data
Feb. 7, 2006 (KR) 10-2006-0011606

(51) Int. Cl. A61B 5/00

(52) U.S. Cl. 600/473

(57) ABSTRACT

A portable body fat measurement apparatus using a near infrared ray, the apparatus including a near infrared sensor to receive a second near infrared ray reflected from a body part of a user after the body part is irradiated with a first near infrared ray, and to convert the second near infrared ray into an electrical signal, an alternating current signal extraction unit to extract an alternating current component from the electrical signal, and a body fat measurement control unit to compare an amplitude of the alternating current component with a predetermined threshold, and to generate an alarm signal when the amplitude of the alternating current component meets the threshold.

### CROSS-REFERENCE TO RELATED APPLICATIONS

This application claims priority from Korean Patent Application No. 10-2006-0011606, filed on Feb. 7, 2006, in the Korean Intellectual Property Office, the disclosure of which is incorporated herein by reference.

### BACKGROUND

1. Field

One or more embodiments of the present invention relate to a body fat measurement apparatus, method, and medium using a near infrared (NIR) signal. More particularly, one or more embodiments of the present invention relate to a body fat measurement apparatus, method, and medium which senses disturbances, such as disturbances generated by a shaking hand of an operator, via an alternating component of an NIR signal reflected from a body part of a user, for example, and generates a predetermined alarm signal to the user when the disturbance is detected to be serious, thereby providing precise and convenient body fat measurement.

2. Description of the Related Art

Obesity rates are on the rise due to increased standards of living and a lack of exercise among the general populace. Obesity may cause many kinds of adult diseases, and even result in discrimination. Accordingly, there is an enhanced interest in diet, and in the treatment and prevention of obesity. In this respect, a person, for example, could recognize his or her own level of obesity by measuring body fat thickness and then determine the need to go on a diet.

Generally, methods of measuring body fat include hydrodensitometry, bioelectrical impedance analysis (BIA), ultrasound assessment of fat, x-ray assessment and near infrared absorption assessment.

Among the described measurement methods, near infrared absorption (“NIR”) assessment is often preferred because of its precision, simplicity and convenience. In the near infrared absorption assessment of fat, body fat is measured using the principal that an NIR ray irradiated into in-vivo tissue is reflected from the in-vivo tissue.

FIG. 1 illustrates such a conventional body fat measurement apparatus using NIR.

Referring to FIG. 1, this NIR body fat measurement apparatus includes an NIR sensor unit including a light emitting sensor 111 and a light receiving sensor 112, an amplifier 120, a partial body fat measurement unit 130, an A/D converter 140, a central processing unit CPU 150, and a driver 160. When the light emitting sensor 111 irradiates a body part of a user, for example, with an NIR ray, a part of the NIR ray is absorbed into the body of the user, and another part may be reflected and received by the light receiving sensor 112. There, the light emitter sensor 111 operates under control of the CPU 150 and the driver 160.

The NIR ray received by the light receiving sensor 112 is converted into an electrical signal to be amplified via the amplifier 120 and forwarded to the partial body fat measurement unit 130. The partial body fat measurement unit 130 extracts a low frequency component of the NIR ray via a low pass filter LPF 131, and then the a low frequency component of the NIR signal is converted into a digital signal via the A/D converter 140 and forwarded to the CPU 150.

The CPU 150 calculates an intensity of the NIR ray reflected from the body of the user by using the NIR signal converted into the digital signal. The CPU 150 may calculate body fat thickness of a body part of a user, for example, by calculating a ratio of the intensity of the reflected NIR ray and an intensity of the NIR ray radiated into the body part of the user via the light emitting sensor 111.

As described above, the NIR body fat measurement unit is widely used as a portable body fat measurement apparatus because of its convenience and simplicity. In NIR body fat measurement, to improve the precision of the measurement, the amount of an NIR ray radiated into a measurement region by the measurement apparatus has to be uniform with respect to all irradiated regions and an NIR irradiation amount, with respect to the region, also has to be uniform at every point in time of measuring.

The amount of NIR radiation radiated by the light emitting sensor 111 may be maintained uniform by the CPU 150 and the driver 160. However, even when the NIR radiation is uniformly radiated via the light emitting sensor 111, when a disturbance occurs, due to a user operating the sensor with a shaking hand, or the trembling of a body part being measured, for example, the NIR ray can not uniformly irradiate the body part of the user. When the NIR ray does not uniformly irradiate a body part due to the disturbance, and the measurement apparatus irradiating the body part is also affected, body fat thickness can not be precisely calculated.

Accordingly, when body fat can not be precisely measured due to a disturbance such as the one described above, a body fat measurement apparatus capable of sensing such a disturbance, notifying a user, and enabling the user to manage the disturbance is required.

### SUMMARY

One or more embodiments of the present invention provides an NIR body fat measurement apparatus, method, and medium in which a disturbance, such as a shaking of a hand of a user, is sensed via an alternating component of an NIR signal reflected and received from a body part of a user, for example, and a predetermined alarm signal is generated and provided to the user when the disturbance is more than a certain level to enable the user to control an occurrence of the disturbance, thereby preventing an error in body fat measurement caused by the disturbance.

One or more embodiments of the present invention also provides an NIR body fat measurement apparatus, method, and medium in which the NIR body fat measurement apparatus is included in a portable device so that a user can conveniently and precisely measure body fat of the user at any time and anywhere to prevent/monitor obesity.

Additional aspects and/or advantages of the invention will be set forth in part in the description which follows and, in part, will be apparent from the description, or may be learned by practice of the invention.

To achieve at least the above and/or other aspects and advantages, embodiments of the present invention include a portable body fat measurement apparatus, using a near infrared ray, including a near infrared sensor to receive a second near infrared ray reflected from a body part of a user after the body part is irradiated with a first near infrared ray, and to convert the second near infrared ray into an electrical signal, an alternating current signal extraction unit to extract an alternating current component from the electrical signal, and a body fat measurement control unit to compare an amplitude of the alternating current component with a predetermined threshold, and to generate an alarm signal when the amplitude of the alternating current component meets the threshold.

To achieve at least the above and/or other aspects and advantages, embodiments of the present invention include a near infrared body fat measurement method, using a near infrared ray, including receiving a second near infrared ray reflected from a body part of a user after the body part is irradiated with a first near infrared ray, converting the second near infrared ray into an electrical signal, extracting an alternating current component from the electrical signal, comparing an amplitude of the alternating current component with a predetermined threshold, and generating an alarm signal when the amplitude of the alternating current component meets the threshold

To achieve at least the above and/or other aspects and advantages, embodiments of the present invention include at least one medium comprising computer readable code to control at least one processing element to implement a method for measuring body fat including receiving a second near infrared ray reflected from a body part of a user after the body part is irradiated with a first near infrared ray, converting the second near infrared ray into an electrical signal, extracting an alternating current component from the electrical signal, comparing an amplitude of the alternating current component with a predetermined threshold, and generating an alarm signal when the amplitude of the alternating current component meets the threshold.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 illustrates a prior art body fat measurement apparatus using NIR, according to an embodiment of the present invention;

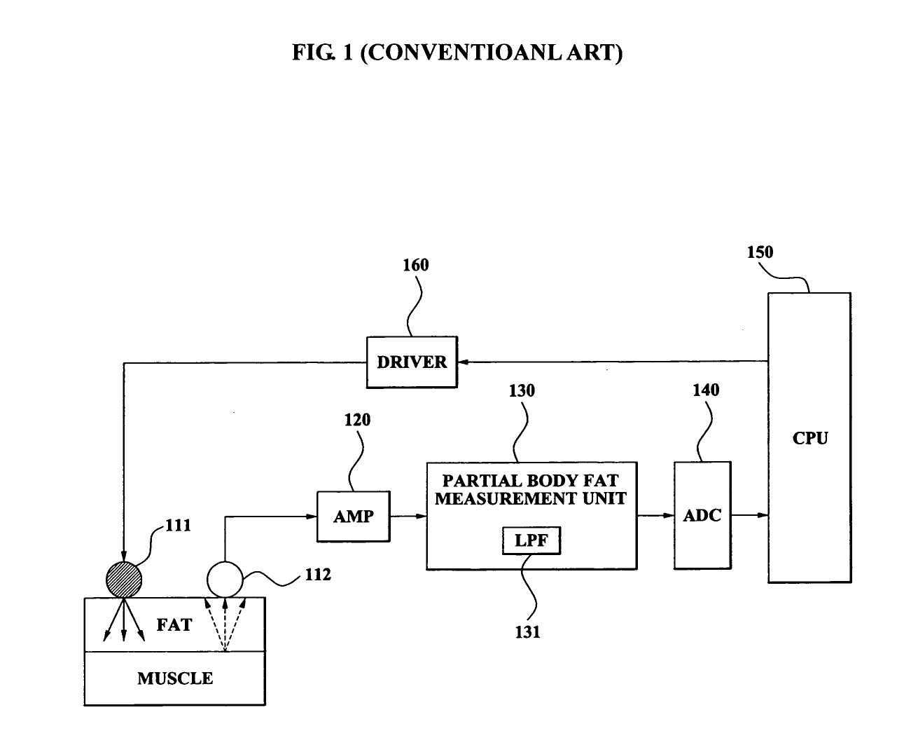

FIG. 2 illustrates a body fat measurement apparatus, according to an embodiment of the present invention;

FIGS. 3(a) and 3(b) illustrate an NIR alternating current signal extracted by an alternating current signal extraction unit, according to an embodiment of the present invention;

FIG. 4 illustrates correlation between a wavelength and an absorption rate of an NIR ray irradiated to a body part of a user, for example, according to an embodiment of the present invention; and

FIG. 5 illustrates an NIR body fat measurement method, according to an embodiment of the present invention.

### DETAILED DESCRIPTION OF EMBODIMENTS

Reference will now be made in detail to embodiments of the present invention, examples of which are illustrated in the accompanying drawings, wherein like reference numerals refer to the like elements throughout. Embodiments are described below to explain the present invention by referring to the figures.

A body fat measurement apparatus, according to an embodiment of the present invention, may be designed to have a stand-alone configuration, or the apparatus may be installed in a portable device such as a mobile communication device, a PDA, a portable game device, an MP3 player, a PMP, a DMB device, and a notebook computer, for example.

Also, the measurement techniques described herein are not limited to a human body. The portable body fat measurement device, according to one or more embodiments of the present invention, may be used for all organisms having a subcutaneous fat layer between skin and muscle.

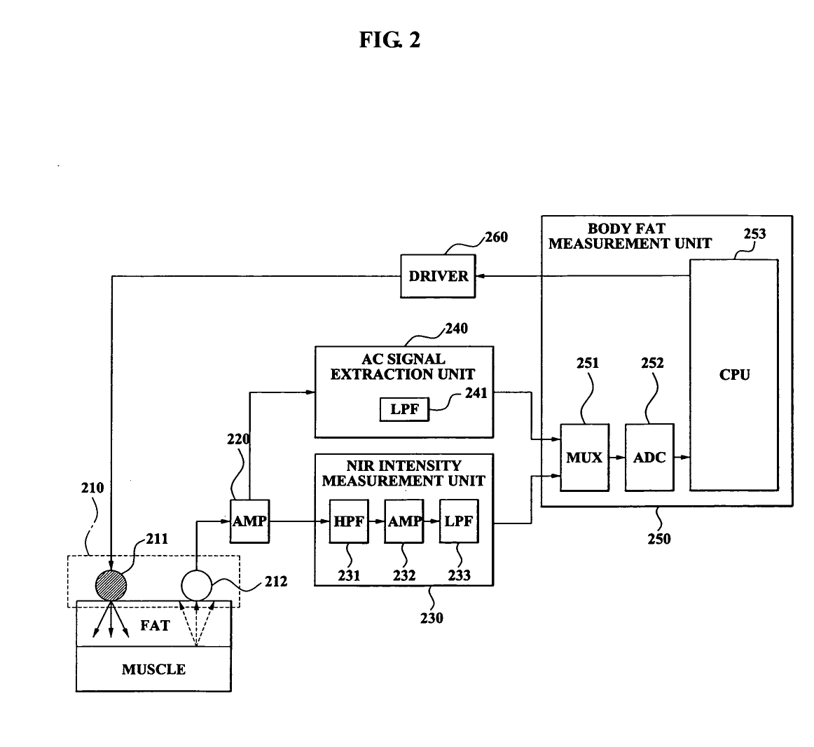

FIG. 2 illustrates a body fat measurement apparatus, according to an embodiment of the present invention. The body fat measurement apparatus may include a near infrared (NIR) sensor unit 210, a first amplifier 220, an alternating current (AC) signal extraction unit 230, an NIR intensity measurement unit 240, a body fat measurement control unit 250, and a driver 260, for example. Here, the NIR sensor unit 210 may include a light emitting sensor 211 and a light receiving sensor 212, the AC signal measurement unit 230 may include a high pass filter (HPF) 231, a second amplifier 232, and a low pass filter (LPF) 233; the body fat measurement control unit 250 may include a multiplexer 251, an analog/digital (A/D) converter 252, and a central processing unit (CPU) 253, also as examples.

The NIR sensor unit 210 may irradiate a predetermined body part of a user with a first NIR ray and generate an NIR signal by converting a second NIR ray reflected from the body part into an electrical signal. As described above, the NIR sensor unit 210 may include the light emitting sensor 211 and the light receiving sensor 212. Namely, in an embodiment, the light emitting sensor 211 irradiates a body part with a first NIR ray according to the control of the CPU 253 and the driver 260. The body part may be any particular body part of a person, for example, for which the user wants to measure body fat thickness, including, but not limited to, an abdomen, thigh, buttocks, upper arm, and calf.

The first NIR ray radiated from the first light emitting sensor 211 may be maintained at a particular intensity by the CPU 253, for example, as it uniformly irradiates a body part of the user.

The first NIR ray radiated by the light emitting sensor 211 may pass through body fat of the body part of the user, with a portion of the first NIR ray being absorbed by muscle while another portion is reflected by the muscle. The reflected NIR ray may be received by the light receiving sensor 212, with the light receiving sensor 212 receiving and converting the reflected NIR ray (hereinafter, referred to as the second NIR ray) into an electrical signal. The second NIR ray (hereinafter, referred to as an NIR signal) may, thus, be converted into an electrical signal and is forwarded/transmitted to the first amplifier 220, for example.

The first amplifier 220 may amplify the NIR signal to be more than a predetermined value and forward/transmit the NIR signal to the AC signal extraction unit 230 and to the NIR intensity measurement unit 240.

As described above, the AC signal extraction unit 230 may include the HPF 231, the second amplifier 232, and the LPF 233, for example.

In an embodiment, the HPF 231 extracts a high frequency (HF) signal from the NIR signal. Namely, the HPF 231 may extract an HF component from the NIR signal. Accordingly, the HPF 231 may generate an NIR AC signal by extracting an AC component of the NIR signal.

Here, the second amplifier 232 receives the NIR AC signal that is generated from the extracted HF component from the HPF 231, amplifies the NIR AC signal to be more than a predetermined value, and transmits the amplified NIR AC signal to the LPF 233.

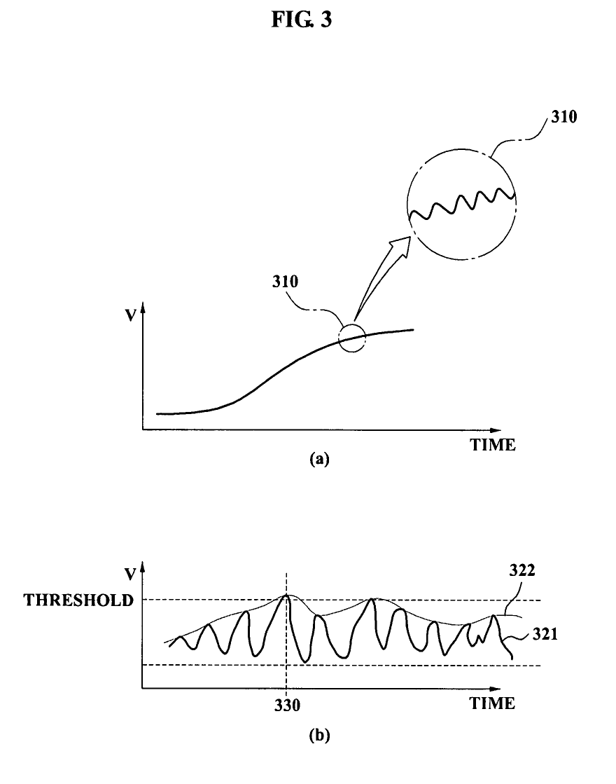

The LPF 233 may further extract a low frequency (LF) signal of the amplified NIR AC signal. Namely, the LPF 233 may extract an LF component from the amplified NIR AC signal. The LF component may indicate a direct current (DC) component of the amplified NIR AC signal, as will be described in greater detail with reference to FIG. 3.

FIG. 3 illustrates an NIR alternating current signal extracted by an alternating current signal extraction unit, according to an embodiment of the present invention.

As shown in FIG. 3(a) the NIR signal amplified by the first amplifier 220 and forwarded/transmitted to the AC signal extraction unit 230 may be an analog DC signal. Such a DC signal does not include an AC signal 310 that is an HF component.

The HPF 231 may extract the AC signal 310, which is the HF component from the NIR signal. In an embodiment, the extracted NIR AC signal may have a waveform 321, as shown in FIG. 3(b). In this embodiment, the LPF 233 may extract an LF component 322 of the NIR AC signal 321, i.e., to compare amplitude of the NIR AC signal 321 with a predetermined value, namely a threshold, and the LPF 233 may extract the LF component 322 from the NIR AC signal 321. Such a comparing of the threshold with the amplitude of the NIR AC signal 321 will be described in greater detail below.

Referring to FIG. 2, the body fat measurement control unit 250 may receive the NIR AC signal 321, as the LF component from the AC signal extraction unit 230. As described above, the body fat measurement control unit 250 may include the multiplexer 251, the A/D converter 252, and the CPU 253, for example.

In an embodiment, the multiplexer 251 receives an NIR AC signal from the AC signal extraction unit 240 and an NIR intensity signal from the NIR intensity measurement unit 230 and forwards/transmits the NIR AC signal and the NIR intensity signal to the A/D converter 252. The multiplexer 251 may select one input signal from a plurality of input circuits and direct the input signal to an output circuit. The NIR intensity signal received from the NIR intensity measurement unit 240 will be described in greater detail further.

The A/D converter 252 may convert the analog NIR AC signal received from the multiplexer 251 into a digital signal, e.g., in order to measure the amplitude of the NIR AC signal.

Here, the CPU 253 may measure, and compare the amplitude of the converted digital NIR AC signal with a predetermined threshold. The amplitude of the NIR AC signal may be measured using a variance or a standard deviation, for example. Namely, the CPU 253 may calculate variance or standard deviation with respect to the amplitude of the LF component of the NIR AC signal and may compare the variance, or standard deviation, with the threshold, noting that alternative embodiments are equally available.

Generally, an NIR body fat measurement apparatus is placed in contact with a body part of a user, for example, while radiating an NIR ray, to begin body fat measurements. If the body part trembles/moves during measurements, or if an operator's hand shakes/moves while holding the body fat measurement apparatus, a change may occur in an amplitude of the NIR AC signal. For example, as shown in FIG. 3(b), in a measurement section in which the shaking or the trembling occurs, the amplitude of the NIR AC signal may be measured to be greater than a predetermined value, or threshold. Generally, when no shaking or trembling occurs, the amplitude of the NIR AC signal will not exceed the threshold.

The threshold may be established at a value sufficiently high such that if the amplitude of the NIR AC signal exceeds the threshold, a disturbance may be determined significant enough to result in inaccurate body fat measurements. The threshold may also be set such that general or typical use, where no significant shaking or trembling occurs, will not result in an NIR AC signal amplitude sufficient to exceed the threshold. Setting the threshold at a suitable value avoids annoying or excessive false alarms while alerting a user to a disturbance of sufficient magnitude to result in imprecise measurements. Alternative factors for defining the threshold are also available.

When the amplitude of the NIR AC signal is measured and fails to meet the threshold, e.g., is greater than the threshold, as a result of comparing the amplitude of the NIR AC signal with the threshold, the CPU 253 may generate a predetermined alarm signal. For example, when the amplitude of the NIR AC signal is measured and is greater than the threshold, the CPU 253 may determine that the body fat thickness cannot be measured precisely due to the movement, e.g., a shaking hand of an operator or the trembling of a body part of the body being measured. Here, the CPU 253 may generate an alarm signal indicating that the body fat thickness cannot be precisely measured.

The CPU 253 may direct the alarm signal to be displayed, sounded, or operated via a predetermined display unit, a sound output unit, a vibrating unit, or a light emitting unit respectively. For example, when the body fat measurement apparatus, according to an embodiment, is included as a component in a mobile communication device, within a system, or as a stand-alone device, the CPU 253 may display the alarm signal on a display screen of the device as another display within the system. Similarly, the CPU 253 may sound the alarm signal via a device speaker. The CPU 253 may also direct the device to vibrate, for example, when set by the user to a silent mode, thereby providing the alarm signal to the user with minimal audible sound. The CPU 253 may also direct a light emitting unit of the device to flash or illuminate as an alarm signal, again as only an example.

When the amplitude of the NIR AC signal is measured and fails to meet the threshold, e.g., is less than the threshold, as the result of comparing the amplitude of the NIR AC signal with the threshold, the CPU 253 may calculate the body fat thickness of the body part by calculating a ratio of intensities of the first NIR ray and second NIR ray. Here, there is either no movement, e.g., the trembling or shaking, on the part of an operator or body, or the trembling or shaking is determined to be insufficient to interfere with the ability of the device to precisely measure body fat. The intensity of the second NIR ray may be measured by the NIR intensity measurement unit 240.

The NIR intensity measurement unit 240 may filter the NIR signal received via the first amplifier 220 via a LPF 241 and may measure an intensity of the filtered NIR signal. Measurement of the NIR intensity may be embodied by including general NIR intensity measurement methods.

In an embodiment, the CPU 253 calculates the thickness of the body fat of the body part by using the below Equation 1.

Thickness of body fat=Kₒ+K₁*(log 1/I) Equation 1

I=Eₛ/Eᵣ

Eₛ: intensity of second NIR ray

Eᵣ; intensity of first NIR ray

Kₒ and K₁: constants

As shown in Equation 1, the CPU 253 may calculate a second value by taking a common logarithm by a reciprocal of a first value made by dividing the second NIR intensity value by the first NIR intensity value, and may calculate the body fat thickness by adding a second constant to a result of multiplying the second value by the first constant, for example. When the amplitude of the NIR AC signal is measured and is greater than the threshold, while calculating the thickness of the body fat, the CPU 253 may stop the body fat measurement operation and may generate an alarm signal and provide the signal to the user.

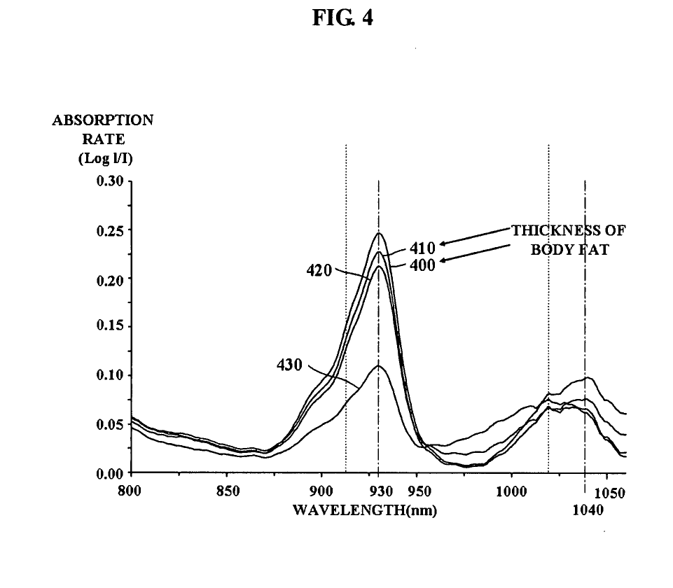

The first NIR ray may have a central wavelength of 930 or 1040 nm, for example, because the thickness of the body fat and an absorption rate of the first NIR ray and the second NIR ray have readily apparent correlation when the NIR ray has these wavelengths. This will be described in greater detail with reference to FIG. 4.

FIG. 4 illustrates the correlation between a wavelength and an absorption rate of an NIR ray used to irradiate a body part of a user, according to an embodiment of the present invention.

Here, in FIG. 4, the body fat thickness of a body part is 10 mm 400, 7 mm 410, 5 mm 420, and 2 mm 430, respectively. Referring to FIG. 4, when a central wavelength of the first NIR ray radiated from a light emitting sensor is 930 or 1040 nm, an NIR absorption rate (log 1/I) with respect to the body part is increased in proportion to the thickness of the body fat.

Accordingly, when irradiating a body part with the first NIR ray having a central wavelength of 930 or 1040 nm, receiving the second NIR ray reflected from the body part, and calculating the NIR absorption rate with respect to the body part utilizing Equation 1 that indicates a proportional relation between the absorption rate, the thickness of the body fat may be derived. Namely, since Equation 1 corresponds to an NIR ray whose central wavelength is 930 or 1040 nm, the first NIR ray may have a central wavelength of 930 or 1040 nm.

The described method of calculating the body fat thickness using the body fat measurement control unit 250 is described for illustrative purposes only, and various kinds of body fat thickness calculation methods, including those conventionally utilized, and as in the present invention, may be used.

As described with reference to FIGS. 2 through 4, when an NIR ray irradiates a body part via an NIR sensor, the NIR body fat measurement apparatus, according to an embodiment of the present invention may precisely detect a disturbance generated by an operator's shaking hand or the trembling of a body part of the person being measured. Namely, not only is the disturbance sensed by using a DC component of an NIR signal, but also an AC component of the NIR signal is separately amplified and used, thereby precisely detecting an occurrence of the disturbance.

The described occurrence sensing operation of the NIR body fat measurement apparatus, according to an embodiment of the present invention, enables a user to detect and control distortion caused by an operator's shaking hand, or the trembling of a body part of the person, for example, being measured, thereby precisely calculating body fat thickness.

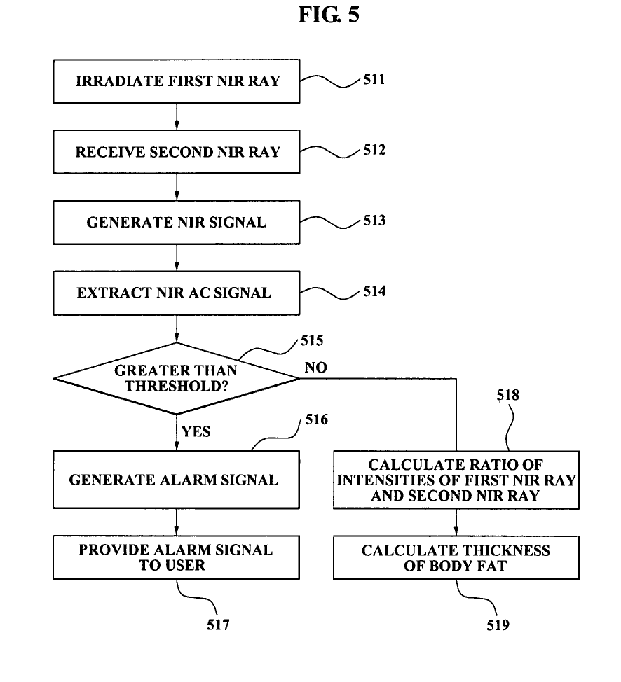

FIG. 5 illustrates an NIR body fat measurement method, according to an embodiment of the present invention.

As shown in FIG. 5, a predetermined body part may be irradiated, in operation 511, using a first NIR ray. A second NIR ray may be reflected from the body part, in operation 512, and an NIR signal may be generated by converting the second NIR ray into an electrical signal, in operation 513.

An AC component may be extracted from the NIR signal, in operation 514. Here, an HF signal may be extracted from the NIR signal, the HF signal amplified, and an LF signal may be extracted from the amplified HF signal, thereby extracting the NIR AC component.

The amplitude of the extracted NIR AC signal may be measured and compared with a predetermined threshold, in operation 515. Here, when, after comparison, the amplitude of the NIR AC signal meets the threshold, e.g., is more than the threshold, a predetermined alarm signal may be generated, in operation 516. The predetermined alarm signal may be constructed to be displayed, sounded, or operated via one or more of a predetermined display unit, a sound output unit, a vibrating unit, and a light emitting unit, for example, in operation 517.

In 515, when, after comparison, the amplitude of the NIR AC signal fails to meet the threshold, e.g., is less than the threshold, a ratio of the intensities of the first NIR ray and the second NIR ray may be calculated in operation 518 and the body fat thickness of a body part may be calculated using the ratio and Equation 1, for example, in operation 519.

The NIR body fat measurement method, according to an embodiment of the present invention, such as that described with reference to FIG. 5, may be performed by the NIR body fat measurement apparatus described with reference to FIGS. 2 through 4, for example.

In addition to this discussion, embodiments of the present invention can also be implemented through computer readable code/instructions in/on a medium, e.g., a computer readable medium, to control at least one processing element to implement any above described embodiment. The medium can correspond to any medium/media permitting the storing and/or transmission of the computer readable code.

The computer readable code can be recorded/transferred on a medium in a variety of ways, with examples of the medium including magnetic storage media (e.g., ROM, floppy disks, hard disks, etc.), optical recording media (e.g., CD-ROMs, or DVDs), and storage/transmission media such as carrier waves, as well as through the Internet, for example. Here, the medium may further be a signal, such as a resultant signal or bitstream, according to embodiments of the present invention. The media may also be a distributed network, so that the computer readable code is stored/transferred and executed in a distributed fashion. Still further, as only an example, the processing element could include a processor or a computer processor, and processing elements may be distributed and/or included in a single device.

According to an embodiment of an NIR body fat measurement apparatus, method, and medium, a disturbance such as an operator's shaking hand may be sensed via an alternating component of an NIR signal reflected and received from a body part of a user, and a predetermined alarm signal is generated and provided to the user when the disturbance exceeds a threshold level, enabling the user to detect the disturbance and thereby preventing a body fat measurement error.

According to another embodiment of an NIR body fat measurement apparatus, method, and medium, the NIR body fat measurement apparatus may be embodied in a stand-alone configuration, embodied as a system, or installed within a portable device, even an existing portable device, so that a user can conveniently and precisely measure body fat of the user at any time and anywhere to prevent/monitor obesity.

Although a few exemplary embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A portable body fat measurement apparatus using a near infrared ray, the apparatus comprising:
a near infrared sensor to receive a second near infrared ray reflected from a body part of a user after the body part is irradiated with a first near infrared ray, and to convert the second near infrared ray into an electrical signal;
an alternating current signal extraction unit to extract an alternating current component from the electrical signal; and
a body fat measurement control unit to compare an amplitude of the alternating current component with a predetermined threshold, and to generate an alarm signal when the amplitude of the alternating current component meets the threshold.

2. The apparatus of claim 1, wherein the alternating current signal extraction unit comprises:
a high pass filter to extract a high frequency signal from the near infrared electrical signal;
an amplifier to amplify the high frequency signal; and
a low pass filter to extract a low frequency signal from the amplified high frequency signal,
wherein the body fat measurement control unit compares the amplitude of the low frequency signal with the threshold.

3. The apparatus of claim 1, wherein the body fat measurement control unit calculates at least one of a dispersion and a standard deviation of the amplitude of the low frequency signal and compares at least one of the dispersion and the standard deviation with the threshold.

4. The apparatus of claim 1, wherein the body fat measurement control unit controls the near infrared sensor to irradiate the body part with the first near infrared ray, and calculates a body fat thickness of the body part by calculating a ratio of an intensity of the first near infrared ray and an intensity of the second near infrared ray when the amplitude of the near infrared alternating current component does not meet the threshold.

5. The apparatus of claim 4, wherein the body fat measurement control unit calculates a second value by taking a common logarithm to an inverse number of a first value obtained by dividing a value of the intensity of the second near infrared ray by a value of the intensity of the first near infrared ray and calculates the body fat thickness by adding a second constant to a value obtained by multiplying the second value by a first constant.

6. The apparatus of claim 4, wherein the body fat measurement control unit stops calculating the body fat thickness when the amplitude of the near infrared alternating current signal is measured to meet the threshold while calculating the body fat thickness.

7. The apparatus of claim 1, wherein the first near infrared ray has a peak wavelength that is one of 930 and 1040 nm.

8. The apparatus of claim 1, wherein the body fat measurement apparatus is installed in a portable device that is at least one of a mobile communication terminal, a personal digital assistant, a portable game device, an MP3 player, a Portable Multimedia Player (PMP), a digital multimedia broadcasting terminal, and a notebook computer.

9. A near infrared body fat measurement method using a near infrared ray, the method comprising:
receiving a second near infrared ray reflected from a body part of a user after the body part is irradiated with a first near infrared ray;
converting the second near infrared ray into an electrical signal;
extracting an alternating current component from the electrical signal;
comparing an amplitude of the alternating current component with a predetermined threshold; and
generating an alarm signal when the amplitude of the alternating current component meets the threshold.

10. The method of claim 9, wherein the extracting of the near infrared alternating current component from the near infrared electrical signal comprises:
extracting a high frequency signal from the near infrared electrical signal;
amplifying the high frequency signal; and
extracting a low frequency signal from the amplified high frequency signal,
wherein the comparing the amplitude of the near infrared alternating current component with the predetermined threshold comprises comparing an amplitude of the low frequency signal with the threshold.

11. The method of claim 9, further comprising, when the amplitude of the near infrared alternating current signal fails to meet the threshold as a result of the calculation, calculating a body fat thickness of the body part by calculating a ratio of an intensity of the first near infrared ray and an intensity of the second near infrared ray.

12. At least one medium comprising computer readable code to control at least one processing element to implement a method for measuring body fat, the method comprising:
receiving a second near infrared ray reflected from a body part of a user after the body part is irradiated with a first near infrared ray;
converting the second near infrared ray into an electrical signal;
extracting an alternating current component from the electrical signal;
comparing an amplitude of the alternating current component with a predetermined threshold; and
generating an alarm signal when the amplitude of the alternating current component meets the threshold.

13. The apparatus of claim 1, wherein the body fat measurement control unit generates the alarm signal to be displayed to the user, sounded, or operated, via at least one of a display unit, a sound output unit, an oscillator, and a light-emitting unit.

14. The method of claim 9, further comprising generating the alarm signal to be displayed to the user, sounded, or operated, via at least one of a display unit, sound output unit, an oscillator, and a light-emitting unit.

15. The medium of claim 12, further comprising generating the alarm signal to be displayed to the user, sounded, or operated, via at least one of a display unit, sound output unit, an oscillator, and a light-emitting unit.

16. The medium of claim 12, wherein the extracting of the near infrared alternating current component from the near infrared electrical signal comprises:
extracting a high frequency signal from the near infrared electrical signal;
amplifying the high frequency signal;
and extracting a low frequency signal from the amplified high frequency signal,
wherein the comparing the amplitude of the near infrared alternating current component with the predetermined threshold comprises comparing an amplitude of the low frequency signal with the threshold.

17. The medium of claim 12, further comprising, when the amplitude of the near infrared alternating current signal fails to meet the threshold as a result of the calculation, calculating a body fat thickness of the body part by calculating a ratio of an intensity of the first near infrared ray and an intensity of the second near infrared ray.
